(19) United States
(12) Plant Patent Application Publication
(10) Pub. No.: US 2003/0233692 P1
(43) Pub. Date: Dec. 18, 2003

(54) Lomandra plant named 'LM300'

(76) Inventor: Todd Anthony Layt, New South Wales (AU)

Correspondence Address: MYERS BIGEL SIBLEY & SAJOVEC, PO BOX 37428, RALEIGH, NC, 27627, US

(21) Appl. No.: 10/402,494

(22) Filed: Mar. 28, 2003

Related U.S. Application Data
(60) Provisional application No. 60/368,198, filed on Mar. 28, 2002.

(51) Int. Cl. A01H 5/00

(52) U.S. Cl. PLT/263

(57) ABSTRACT

A new and distinct variety of Lomandra longifolia plant, designated ‘LM300’, is characterized by a fine, narrow leaf blade, and compact habit as compared with other varieties of Lomandra longifolia. ‘LM300’ is further characterized by a green to deep-green leaf blade color with purple/dark brown basal shoots. In addition, ‘LM300’ is cold tolerant with excellent color retention as compared with other varieties of Lomandra longifolia.

### RELATED APPLICATION INFORMATION

This application claims the benefit of U.S. Provisional Application Serial No. 60/368,198 filed 28 Mar. 2002, the disclosure of which is incorporated herein by reference in its entirety.

### LATIN NAME OF THE GENUS AND SPECIES

The Latin name of the novel variety disclosed herein is Lomandra longifolia.

### VARIETY DENOMINATION

The inventive variety of Lomandra disclosed herein has been given the variety denomination ‘LM300’.

### BACKGROUND OF THE INVENTION

The present invention relates to a new and distinct variety of perennial Lomandra longifolia, which has been named ‘LM300’. Lomandra are a genus of ornamental grass-like plants. The variety ‘LM300’ was discovered in a nursery in April 1998 in the state of New South Wales, Australia, during a routine inspection of large quantities of cultivated Lomandra longifolia ‘Katrinus’ (Australian Plant Breeders Rights Application No. 1997/168) production stock. ‘LM300’ is believed to be an “off type” or sport of Lomandra longifolia ‘Katrinus’, due to its much finer leaf and compact size as compared with Lomandra longifolia ‘Katrinus’. The new variety ‘LM300’ was first propagated asexually by division in August 1999 in the state of New South Wales, Australia, and has since been asexually propagated by division and tissue culture. The distinctive characteristics of the inventive variety are stable from generation to generation; clones of the variety produced by asexual reproduction maintain the distinguishing characteristics of the original plant.

‘LM300’ has a compact habit and a narrow leaf forming an attractive ornamental grass-like plant, unlike its parent, which is characterized more as a strapy leaf plant.

An application for plant breeders' rights with respect to ‘LM300’ has been lodged with the Australian Plant Breeders Rights Office, and was first gazetted in May 2001 (under Application No. 2001/092).

### SUMMARY OF THE INVENTION

‘LM300’ is a distinctive variety of Lomandra longifolia, which is characterized by its combination of color, compact growth habit, and fine narrow leaf blade as compared with other varieties of Lomandra longifolia. It is a short, rhizomatous plant forming a compact tussock (FIG. 8). The inventive ‘LM300’ variety has a dwarf and more compact growth habit, and leaves which are finer and narrower, as compared with Lomandra longifolia ‘Katrinus’, Lomandra longifolia ‘Cassica’ (Australian Plant Breeders Rights Application No. 1997/166), or common Lomandra longifolia. Lomandra longifolia ‘LM400’ (Australian Plant Breeders Rights Application No. 2001/090; U.S. provisional application 60/400,389) also has a fine, narrow leaf blade and compact growth habit; however, ‘LM300’ may be distinguished from ‘LM400’ in that it has a greener leaf, a less spiky flower spike, and is more compact in habit than ‘LM400’. ‘LM300’ also exhibits cold tolerance with excellent color retention.

### BRIEF DESCRIPTION OF THE DRAWINGS

FIG. 1 shows a comparison in leaf width of ‘LM300’ with several known varieties of Lomandra longifolia plants, from left to right, common Lomandra longifolia, ‘Katrinus’, ‘Cassica’, ‘LM400’ and ‘LM300’.

FIG. 2 provides a comparison of the length of the leaf of ‘LM300’ with several known varieties of Lomandra longifolia plants, from left to right, common Lomandra longifolia, ‘Katrinus’, ‘Cassica’, ‘LM400’ and ‘LM300’.

FIG. 3 demonstrates the wider leaf of ‘LM300’ as compared with ‘LM400’.

FIG. 4 shows a comparison between the basal shoots of ‘LM400’ (left) and ‘LM300’ (right).

FIG. 5 provides a comparison of the seed head of ‘LM300’ with several known varieties of Lomandra longifolia plants, from left to right, ‘Katrinus’ ‘Cassica’, ‘LM400’, and ‘LM300’.

FIG. 6 shows another comparison between the basal shoots of ‘LM400’ (left) and ‘LM300’ (right).

FIG. 7 shows a comparison between LM300 (left) and LM400 (right) plants.

FIG. 8 shows a LM300 plant grown in a one-gallon pot.

FIG. 9 and FIG. 10 show a LM300 plant in a landscape north of Atlanta, Ga. after a temperature of 16° F. (−8.9° C.) was recorded.

### DETAILED BOTANICAL DESCRIPTION OF THE VARIETY

The following is a detailed botanical description of a new and distinct variety of Lomandra longifolia known as ‘LM300’, based upon observations of the plant grown in nursery pots and field plots in New South Wales, Australia, unless otherwise indicated. The data presented in Tables 1-3, below, were obtained from mature plants that were approximately eighteen months old on Jul. 6, 2001 when the trials commenced. Those skilled in the art will appreciate that certain characteristics will vary with older or, conversely, with younger plants. ‘LM300’ has not been observed under all possible environmental conditions. Where dimensions, sizes, colors and other characteristics are given, it is to be understood that such characteristics are approximations or averages set forth as accurately as practicable. The phenotype of the variety may differ from the descriptions herein with variations in the environment such as season, temperature, light intensity, day length, cultural conditions, and the like. Color notations are based on The Royal Horticultural Society Colour Chart, The Royal Horticultural Society, London, 1995 edition.

‘LM300’ is a perennial, vegetatively propagated Lomandra longifolia plant, and is believed to be an “off type” or sport of Lomandra longifolia ‘Katrinus’. ‘LM300’ is a narrow-bladed compact plant, which is unusual for Lomandra longifolia, as these plants are usually medium- to wide-bladed strapy plants, and are larger in overall size than ‘LM300’. A description of the distinguishing characteristics of ‘LM300’ and comparisons with other varieties of Lomandra longifolia are provided in the accompany figures, in Tables 1-3, and in the discussion below.

### Technical Description of the Variety

Plant characteristics: Growth habit upright, height short, distal weeping present, proliferation rhizomatous, depth of rhizomes deep.

Leaves: Color green (RHS 138A), surface glabrous, attitude upright, apex indentation present, number of predominant indentation two, length of blade long (mean 557.5 mm), width of blade very narrow (mean 3 mm) for this species, rigidity non-stiff (flexible), texture non-coriaceous, curvature concave to convex near base and nearly flat at apex (generally thickened and more incurving on the right hand abaxial edge). The leaf blade is generally a uniform width from base to the tip, with the jagged tip typical of Lomandra longifolia.

Basal sheath: Color dark brown, distally tapering, usually tattered.

Basal Shoots: Texture fine, width narrow, attitude upright, arrangement cluster. The basal shoots are purple/brown in color.

Inflorescence: Branching present (generally 2 at nodes), arrangement whorled, shape of scape in cross-section oval at base and rectangular distally. The inflorescence (flower spike) is yellow with an average length of 195 mm, and an average width of 25 mm. The flower spike is non-spiky to touch, which is unusual for this species.

Bracts: Color transparent to straw colored, position of bracts at the base of each flower cluster, length of bracts in comparison to flower cluster longer, sharp pointed.

Flowers: Color of outer perianth off-white, color of inner perianth cream-yellow.

Roots: Similar to other Lomandra longifolia, ‘LM300’ has a massive root structure.

These and other features and characteristics of ‘LM300’ are apparent from the figures provided herein.

The growth and morphology of ‘LM300’ is further characterized in Table 1, which presents data regarding leaf blade width, blade length, leaf color, flower spike length, ‘spikiness’ of the flower spike, and compactness.

### Environmental Tolerances

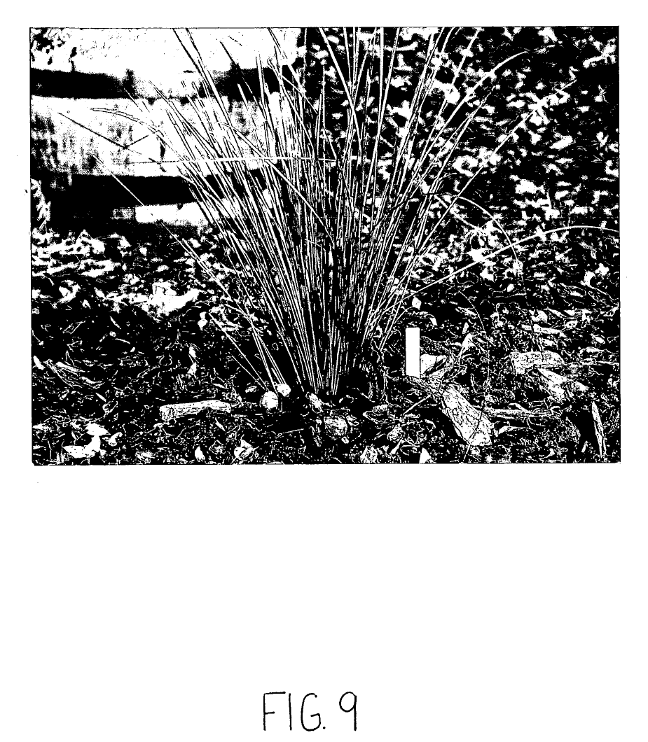

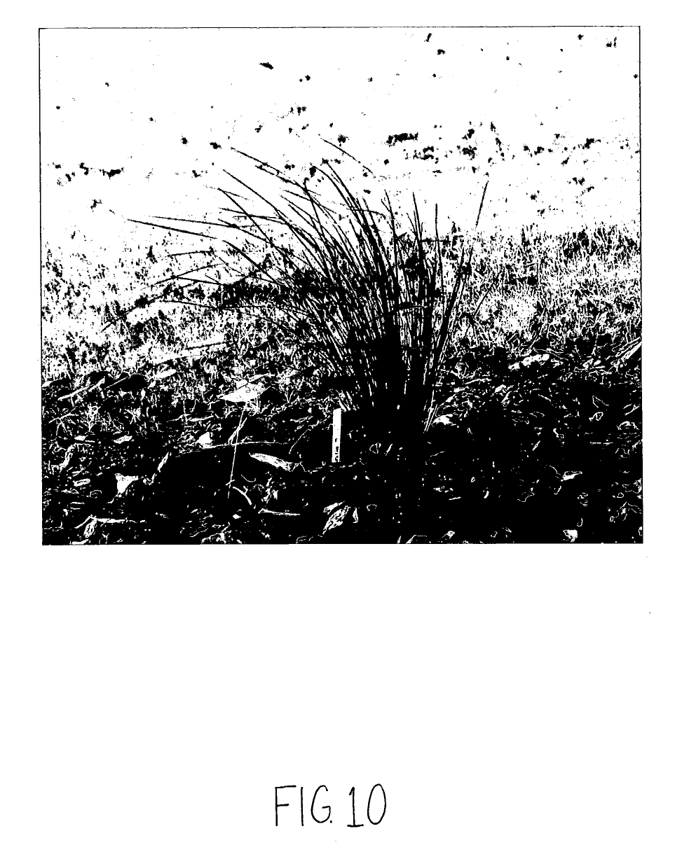

‘LM300’ has shown potential for shade tolerance and further shade tolerance tests are underway. ‘LM300’ has excellent color retention in winter as compared with other varieties of Lomandra longifolia. The winter hardiness of ‘LM300’ is at least to Zone 8a, and evaluation of winter hardiness is ongoing. ‘LM300’ has been observed to hold color to 6° F. (−14.4° C.). FIGS. 9 and 10 show a ‘LM300’ plant growing in the landscape north of Atlanta, Ga. after a temperature was recorded of 16° F. (−8.9° C.). The plant shows good color retention, and other dormant grasses can be seen in the background. Winter color retention is also the subject of ongoing tests.

‘LM300’ has excellent drought tolerance, most likely due to the massive deep root system. ‘LM300’ has been noted to recover with watering, even after severe wilting.

‘LM300’ can grow in most soil types, including sandy, clay, alkaline or acidic soils.

TABLE 1

| | | Basal | Blade Length | | Flower Spike | | |
|---|---|---|---|---|---|---|---|
| | Blade | Shoot | (Av. length | | Length | Flower Spike Width | |
| Species | Width | Membrane | only | Color | (Av. length only) | (Av. width only) | Spikiness |
| LM300’ | 2.5 to | Purple/Dark | 557.5 mm | Green | 195 mm | 25 mm | Very Little |
| | 3.5 mm | Brown | | | | | Spikiness |
| LM400’ | 3 to | White/Green | 714 mm | Blue/Grey | 185 mm | 25 mm | Medium |
| | 5 mm | & Light | | | | | Spikiness |
| | | Brown | | | | | |
| Katrinus’ | 7 to | Purple | 947.5 mm | Green | 240 mm | 65 mm | Medium to |
| | 10 mm | | | | | | Very |
| | | | | | | | Spiky |
| Cassica’ | 10 to | White/Green | 1095 mm | Blue/Grey | 230 mm | 48 mm | Very |
| | 15 mm | | | | | | Spiky |
| Common | 10 to | Variable | 1085 mm | Green- | 240 mm | 51 mm | Very |
| Lomandra* | 16 mm | | | Blue/Grey | | | Spiky |

*Not subject to Australian Plant Breeders Rights
Reference; Test Plots located at Abulk Nursery in Windsor, New South Wales, AUSTRALIA. Planted by Abulk Pty Ltd on Jul. 6, 2001.

### Disease Resistance and Susceptibility

‘LM300’ has a good resistance to root rot as compared with other Lomandra longifolia.

### Comparisons with Other Lomandra longifolia

‘LM300’ may be readily distinguished from other varieties of Lomandra Longifolia.

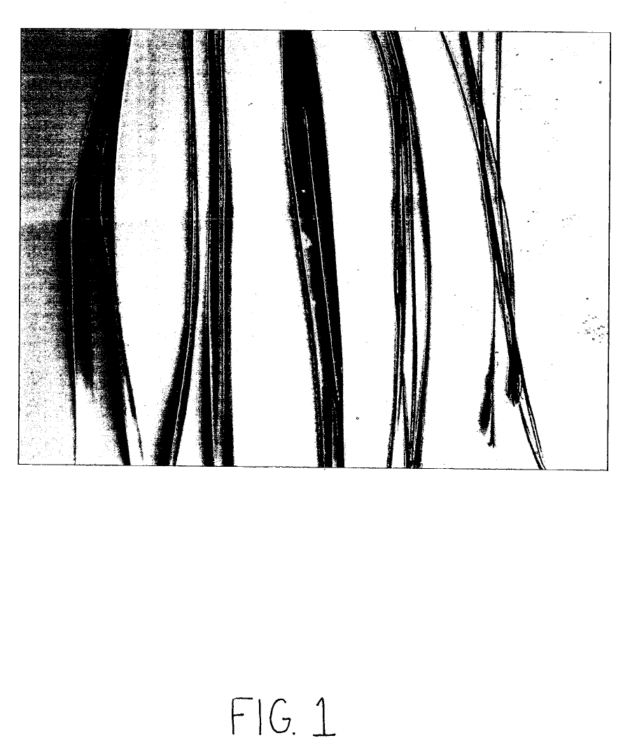

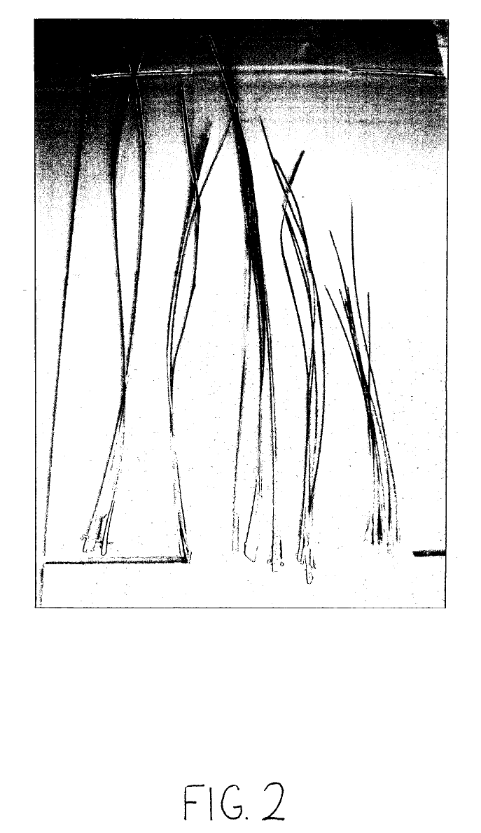

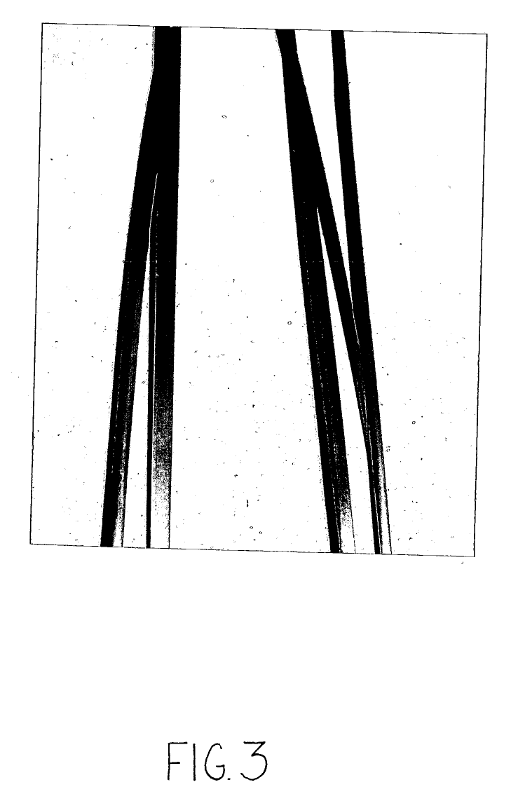

The leaf blade width of ‘LM300’ is finer and narrower than Lomandra longifolia ‘Katrinus’ and other Lomandra longifolia, with the exception of ‘LM400’ (FIGS. 1, 2 and 3).

‘LM300’ has a more compact habit than Lomandra longifolia ‘Katrinus’, Lomandra longifolia ‘Cassica’ and other common forms of Lomandra longifolia.

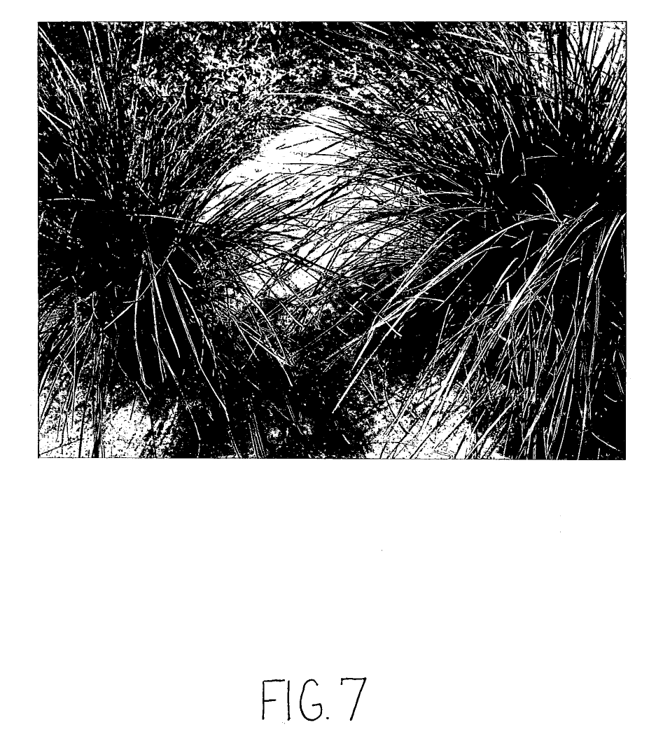

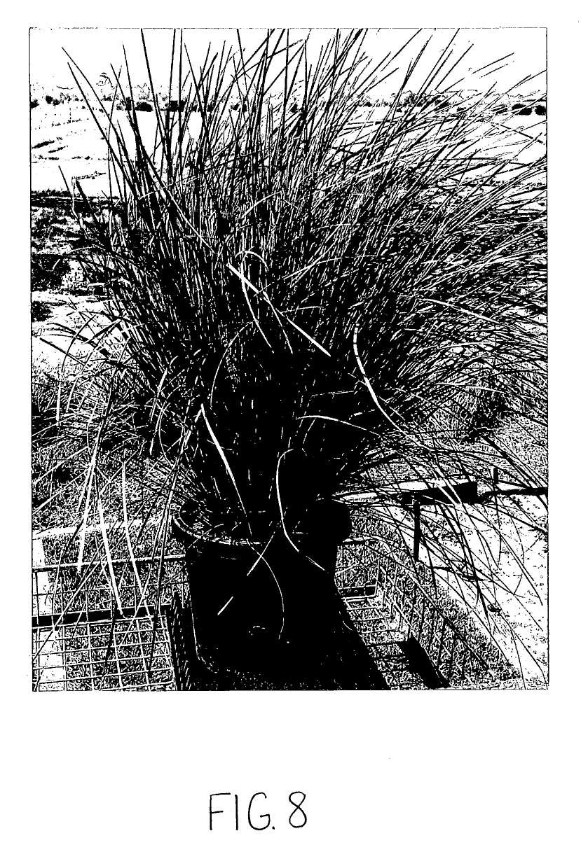

Lomandra longifolia ‘LM400’ also has a narrow blade and a compact habit. ‘LM300’ may be distinguished from ‘LM400’ in that ‘LM300’ has a greener leaf color (FIG. 7), a less spiky flower spike, and a more compact growth habit.

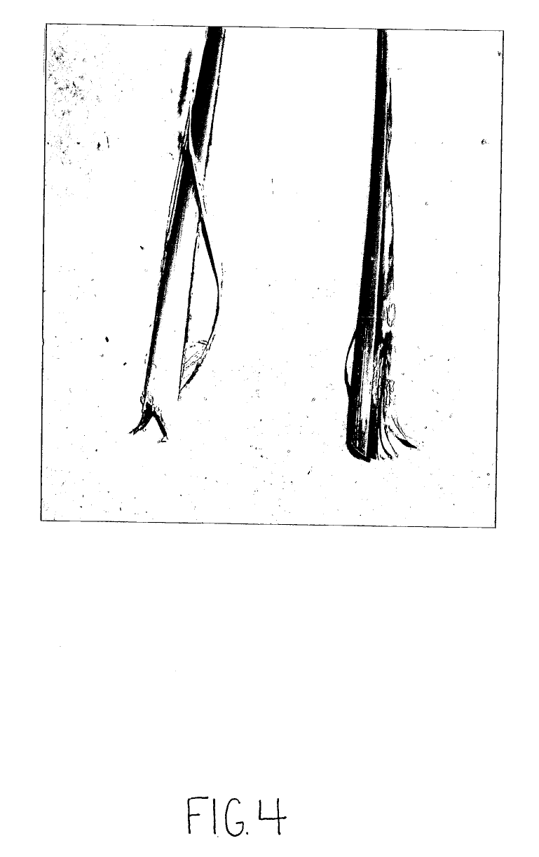

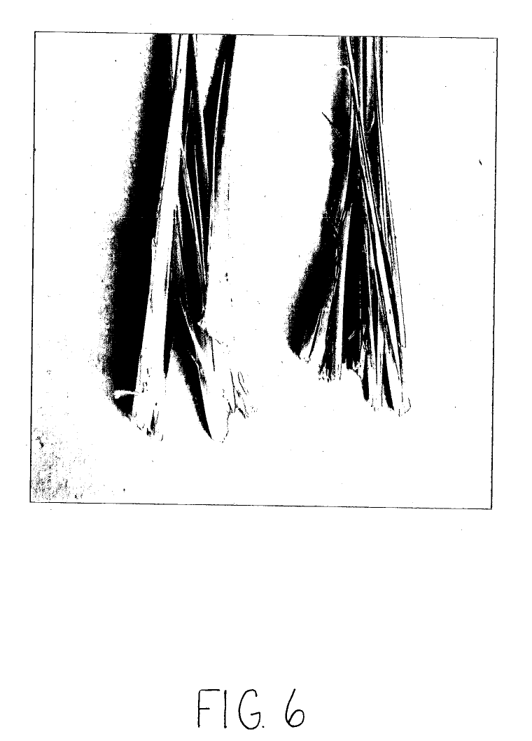

The leaves of most Lomandra are a grey-green to grey-blue color. In contrast, ‘LM300’ is green to deep-green in color. In addition, the basal shoots of ‘LM300’ are purple/dark brown, similar to ‘Katrinus’ which has purple basal shoots (FIGS. 4 and 6). In contrast, ‘LM400’ has grey-blue leaf color and white-green or light brown basal shoots.

‘LM300’ has a flower spike, which is not spiky to touch, whilst the flower spike of Lomandra longifolia ‘Katrinus’ and Lomandra longifolia ‘Cassica’ are relatively spiky to touch. ‘LM300’ is also less spiky to touch than ‘LM400’.

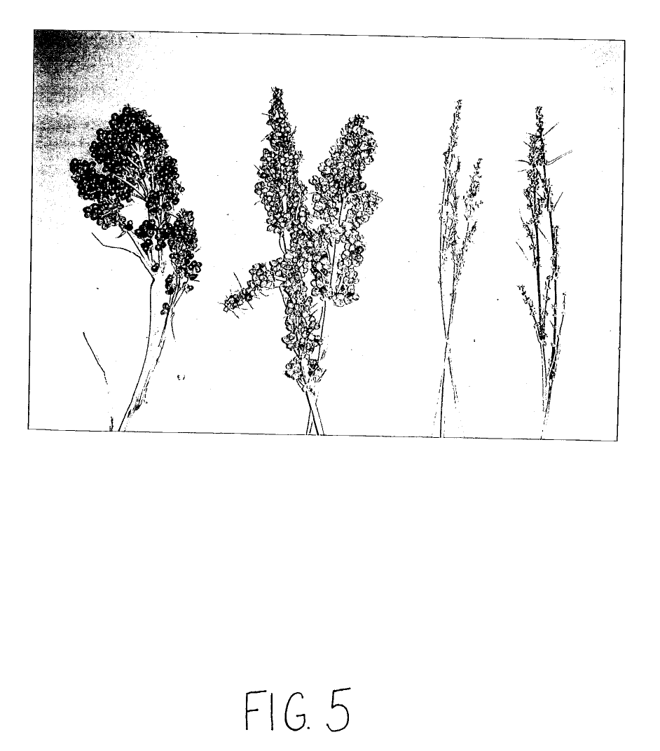

‘LM300’ has a smaller flower spike than Lomandra longifolia ‘Katrinus’ and Lomandra longifolia ‘Cassica’. The flower spike of ‘LM300’ is similar to that of ‘LM400’ in size. Thus far, ‘LM300’ has not been observed to produce viable seed, whilst Lomandra longifolia ‘Katrinus’, ‘Cassica’ and most other known Lomandra longifolia produce viable seed from the female plant (FIG. 5). Like ‘LM300”, ‘LM400’ does not appear to produce viable seed.

### Asexual Reproduction

After its initial discovery, ‘LM300’ was transplanted into a 140 mm pot for further trials and testing. After divisions were made for several subsequent generations, ‘LM300’ was observed to retain the color, size, and fine leaf characteristics that were originally noted in the parent plant. ‘LM300’ was then divided into many larger pots for further evaluation. ‘LM300’ has also been asexually propagated using tissue culture techniques.

During these divisions, it was noted that ‘LM300’ can produce 7 to 10 divisions per 140 mm pot, as compared with 2 to 5 divisions for Lomandra longifolia ‘Katrinus’, Lomandra longifolia ‘Cassica’ and common Lomandra longifolia. More extensive data regarding division rates per 140 mm pot of ‘LM300’ in comparison with other Lomandra longifolia are presented in Table 2.

TABLE 2

| | Divisions | Divisions |
|---|---|---|
| | per 140 mm | per 300 mm |
| Species | Pot (Range) | Pot (Range) |
| ‘LM300’ | 7 to 10 | 24 to 32 |
| ‘LM400’ | 5 to 8 | 22 to 26 |
| ‘Katrinus’ | 4 to 5 | 8 to 13 |
| ‘Cassica’ | 2 to 3 | 6 to 7 |
| Common | 2 to 3 | 6 to 9 |
| Lomandra* | | |

*Not subject to Australian Plant Breeders Rights
Reference; Abulk Nursery test plots at Windsor, New South Wales, AUSTRALIA
Divided between Jul. 6, 2001 and Dec. 15, 2001

Divisions of ‘LM300’ root quickly, within 3 to 4 weeks, as compared with 5 to 7 weeks for the parent Lomandra longifolia ‘Katrinus’. The survival rate for ‘LM300’ division is 99% whilst Lomandra longifolia ‘Katrinus” is approximately 85%. A more detailed comparison of division survival rate is provided in Table 3.

TABLE 3

| | No. Of Divisions That |
|---|---|
| Species | Survived Out of 200 |
| ‘LM300’ | 198 |
| ‘LM400’ | 194 |
| ‘Katrinus’ | 170 |
| ‘Cassica | 159 |
| Common | 163 |
| Lomandra* | |

Not subject to Australian Plant Breeders Rights
Reference; Abulk Nursery test plots at Windsor, New South Wales, AUSTRALIA
Divided between Jul. 6, 2001 and Dec. 15, 2001
50% sand and 50% peat was the potting mix used, with plants being divided into 90 mm × 50 mm × 50 mm tubes.

That which is claimed is:

1. A new and distinct variety of Lomandra longifolia plant named ‘LM300’, substantially as described and illustrated herein.
